(19) United States
(12) Patent Application Publication
(10) Pub. No.: US 2014/0134049 A1
(43) Pub. Date: May 15, 2014

(54) Lateral Flow Assay Device for Measuring Low Quantity Sample

(71) Applicant: Daniel Wang, San Diego, CA (US)

(72) Inventor: Daniel Wang, San Diego, CA (US)

(21) Appl. No.: 13/678,140

(22) Filed: Nov. 15, 2012

(51) Int. Cl. G01N 33/53

(52) U.S. Cl. CPC G01N 33/5302
USPC 422/69

(57) ABSTRACT

This invention describes a design of a lateral flow assay device that detects dried chemicals or trice volume aqueous sample solutions, applicable for detecting body fluids and dried or liquid chemicals. The dried or aqueous samples on the sample loading area will contact with a secondary aqueous solution in described manner and flow to the reaction area. This invention enables a complete lateral flow assay while the sample volume itself is too small to accomplish a complete lateral flow test.

### CLAIM OF PRIORITY

This application claims priority under 35 USC §119(e) to U.S. Patent Application Ser. No. 61/629,345, filed on Nov. 17, 2011, the entire contents of which are hereby incorporated by reference.

Reference Cited
U.S. Patent Documents

| | | |
|---|---|---|
| 4,855,240 | August 1989 | Rosenstein et al. |
| 5,120,643 | June 1992 | Ching, et al. |
| 5,569,608 | October 1996 | Sommer |
| 5,591,645 | January 1997 | Rosenstein |
| 5,656,503 | August 1997 | May et al. |
| 6,303,081 | October 2001 | Mink et al. |
| 7,192,555 | March 2007 | Mink et al. |
| WO/1999/006827 | November 1999 | Fitzpatrick, Judith |

### OTHER REFERENCES

(1) Drummer, 0.; Drug Testing in Oral Fluid, Clin Biochem Rev. 2006 August; 27(3): 147-159
(2) Hofman L.; Human Saliva as a Diagnostic Specimen, J. Nutr. May 1, 2001 vol. 131 no. 5 1621S-1625S
(3) Kaufman E.; Lamster I.; The Disagnostic Applications of Saliva—A Review, CROBM March 2002 vol. 13 no. 2197-212
(4) Loo, J.; Yan, W.; Ramachandran, P.; Wong, D.; Comparative Human Salivary and Plasma Proteomes, J Dent Res. 2010 October; 89(10): 1016-1023
(5) Mittal S, Bansal V, Garg S, Atreja G, Bansal S. The diagnostic role of Saliva—A Review. J Clin Exp Dent. 2011;3(4):e314-20
(6) Nagler R.; Saliva Analysis for Monitoring Dialysis and Renal Function, Clinical Chemistry, 2008, 54:9, 1415-1417
(7) Schramm, W.; Draig, P. A.,; Smith R.; and Berger G.; Cocaine and Benzoylecgonine in saliva, serum, and urine, Clinical Chemistry, March 1993 vol. 39 no. 3 481-487
(8) Shrivastava, S.; Bastian T S, Singh, A.; Jaiswal R.; The use of saliva as an unconventional sample for drug detection: A review, Medico-Legal Update, Vol. 8, No. 2 (2008, July- 2008, Dec.

### FIELD OF THE INVENTION

This invention relates to a device for trice amount analytes detection in the environment, surface of objects and components of life body (sample). The design requires testing of one or multiple analyte sample together with an extra aqueous solution that provides sufficient volume for the test to be complete.

### BACKGROUND OF INVENTION

In the last a few decades, rapid lateral flow assay has been developed for determining the presence or absence of analyte in body fluid, on the surface of objects or in the environment such as blood, urine, saliva and other liquid samples and dried or aqueous chemicals. Multiple patents related to the lateral flow assays were awarded (U.S. Pat. Nos. 4,855,240, 5,120,643, 5,569,608, 5,591,645, 5,656,503, and 6,303,081, 7,192,555). The drugs of abuse (DOA) test is one of the most broadly use test using this rapid lateral flow test platform. Currently, the billion-dollar market of DOA is dominated by rapid lateral flow test using urine samples. The users are clinics (FDA regulated diagnostic device), employers that perform pre-employment screening/random employee testing and government mandated drug testing. The primary benefit of the oral testing is its ability to negate the privacy concerns. The oral testing method can be similar to taking an oral thermometer reading face to face, leaving little chance of sample adulteration by the drug user. Also, the oral fluid testing can detect the parent THC (marijuana), indicating the drug user is under the influence of the drug. Other benefits comprise user-friendly, very convenient for on-site testing, able to repeated sampling. Equally important is the close correlation of the drug concentration in the oral fluid to that in the blood (2, 3, 7).

Saliva is a unique fluid and interest in it as a diagnostic medium has advanced exponentially in the last 10 years (1, 5). In the United States, the need for further research in salivary diagnostics has been emphasized by federal action plans emanating from the Office of the Surgeon General [Health and Human Services (HHS), 2009] and the National Institute of Dental and Craniofacial Research (NIDCR, 2009). The literature is replete with articles, 2500+ since 1982, describing the use of saliva, gingival crevicular fluid, and mucosal transudates for drug monitoring and for the detection of various oral and systemic maladies.

Advances in the use of saliva as a diagnostic fluid have also been tremendously affected by current technological developments. For example, the ability to measure and monitor a wide range of molecular components in saliva and compare them to serum components has made it feasible to study microbes, chemicals and immunologic markers (3, 6). As a consequence, these advances in technology have helped to move saliva beyond measuring oral health characteristics to where it now may be used to measure essential features of overall health.
Demonstrated oral fluid tests in published scientific literatures:
Drugs of Abuse, multiple targets
Autoimmune disorder: Sjogren's syndrome
Cardiovascular marker: amylase, CRP
HIV
Bacteria infections: H. pylori, Streptococcus mutans, Lactobacillus acidophilus, Porphymonas gingivalis
IgG: Measles, Rubella, Hepatitis
Renal disease screening: nephrology, creatinine concentrations
Cancer: EGF in ovarian cancer, breast cancer
Drug monitoring: levels of 3-methoxy-4-hydroxyphenylglycol (MHPG) for psychiatric therapy.
Bio-terror detections: anthrax bacillus and chemical agents

While the oral fluid rapid drug testing has obvious merits and some products were already launched for use in the FDA non-regulated market, there exist technical obstacles that limit its broad applications. For instance, due to the variable nature of saliva in viscosity, dry mouth, age, gender, time of saliva collection, the collection time with currently available collection devices for oral fluid sample can be too long (8). Collecting enough volume of oral fluid for conducting the test run may take more than 5 minutes, yet in many cases collecting enough volume became unsuccessful. Indeed, the needed volume for the sample to mix and bind to its ligand in the mentioned assay platform is often as little as less than 0.05 milliliter, although this is still not enough to maintain the lateral flow by capillary force to complete the test run.

### BRIEF SUMMARY OF THE INVENTION

This invention describes a test design that the need for the sample volume is minimal, and a secondary aqueous solution is applied to maintain the flow while the primary sample is not significantly diluted.so that a lengthy, large volume collection becomes unnecessary and for the dried chemicals, the added aqueous solution also serve as the diluent that permits the dried component to be detected onsite. The small volume requirement allows the sample collection time to be significantly reduced, and minimized the dilution, therefore making the lateral flow assay a much faster and more effective test.

With conventional saliva collection/testing methods, collecting enough volume of oral fluid for conducting the test run usually take minutes. In many cases collecting enough volume became unsuccessful. The needed volume for the sample to mix and bind to its ligand in the mentioned assay platform is often as little as less than 0.05 milliliter, although this is still not enough to maintain the lateral flow by capillary force to complete the test run. Here we describe our invention that the test requests a minimized sample collection pad and a small portion of the one end of the pad is further contacted with an aqueous solution (chasing solution) to accomplish the lateral flow testing, so that it is small enough to reduce the sample collection volume and time and the collected sample volume (or amount for dried samples) will be sufficient for the testing. The minimized overlapping of the sample loading area and the chasing solution enables the sample(s) to reach the reaction area undiluted or minimized diluted therefore will greatly increase test sensitivity and shorten the sample collection time.

### BRIEF DESCRIPTION OF THE DRAWING

FIG. 1 shows an assay set vertically in contact with the secondary aqueous solution at the bottom inside the sample well that holds the assay in a vertical position.

### DETAILED DESCRIPTION

This invention describes a design of a lateral flow assay device that detects dried chemicals or trice volume aqueous sample solutions, applicable for detecting body fluids and dried or liquid chemicals. The dried or aqueous samples on the sample loading area will contact with a secondary aqueous solution in the described manner and flow to the reaction area. This invention enables a complete lateral flow assay while the sample volume itself is too small to accomplish a complete lateral flow test.

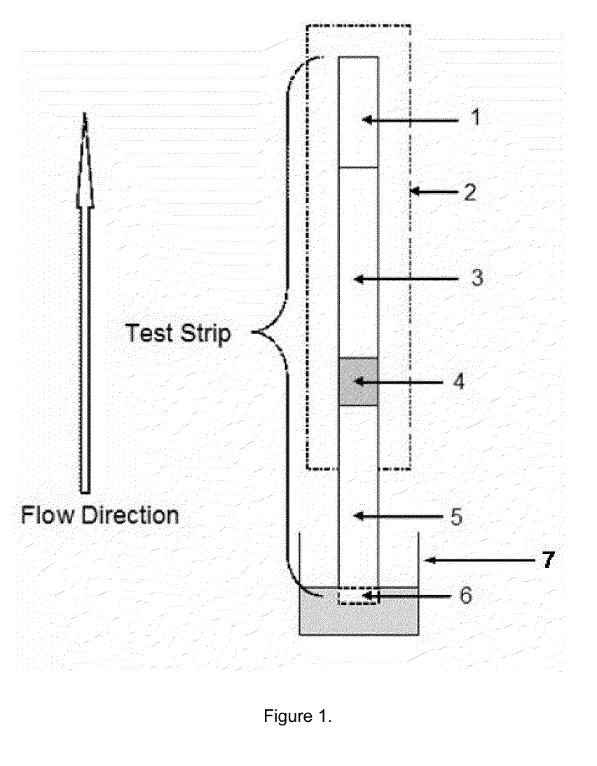

FIG. 1 shows an example of an assay that is tested with minimum contact with secondary solution. The test strip can be directly or indirectly used to obtain a sample with an analyte, and the strip is set vertically, its sample pad 5 at the bottom. The tip 6 of sample pad 5 contacts secondary aqueous solution. The solution will travel up the assay due to capillary force, pushing the analyte from the sample on the sample pad 5 to the binding zone 4, which is where at least one type of tracer that has visual indicator is located. Different analyte has a corresponding binding site to each type of tracer. Once the analyte reaches binding zone 4, each analyte binds to the corresponding tracer and they are pushed into the binding zone 3, where at least one binder material is immobilized. Each binder material is bound to one type of analyte. When tracers reach the binder material, if they are not bound to the analyte, they will bind to the analyte bound to the binder material. If the tracers are bound to the analyte from the sample, then the tracers will pass through, preventing the visual indicator to appear. If the tracers successfully bind to the last binder material, the visual indicator will appear indicating that the tracers successfully travelled through the assay.

The test strip can be contained in a housing 2 for the means of, but not limited to, protecting the test strip and the components that are necessary for running the test, including the tracers and binder materials, writing the dates and information of patients who are being tested, and labeling the binder materials or the analyte that can be tested. The housing 2 allows access to sample pad 5, enabling the collection of a sample.

The assay may include a container 7 that contains the secondary aqueous solution.

The size, material, or structure of the container 7 is not limited to a particular standard, however, the components listed must allow the tip of the sample pad 5 to be in contact with the solution, minimizing the amount of contact but sufficient to run and complete the test. The container 7 may include a structure that holds the assay vertically.

The portion that provides the capillary flow is formed from microfluidic channels or absorbent material including nitrocellulose, glass fiber, cellulose, nylon, and etc. that is capable of transporting a liquid by capillarity. The selection of a suitable material is deemed to be within the scope of those skilled in the art from the teachings herein.

The sample pad is where the analyte, or the molecules that are being detected by the assay, will be placed. The sample pad can be composed of any absorbent or porous material. The analyte can be any molecules, small or large, that is being detected in the assay. It can include, but not limited to, antibodies, drug metabolites, antigens, enzymes, and proteins, and the properties can include, but not limited to, solid, semi-solid, and liquid. Generally, the analyte from a liquid sample will flow on the fibrous and/or porous solid phase support towards a previously resided tracer on the support and then co-flow with the tracer towards the binder resulting in tracer and analyte communication with the binder on the porous support.

Depending on the assay design, each analyte or ligand on the tracer or the binder on the porous support has distinct characteristics and binding specificities.

For example, the analyte can be a small or large molecule as the target for an antibody (or other molecule) to specifically bind to it. In this case, the ligand on the tracer can be the antibody (or other molecule) and the binder on the support membrane can be the same or similar analyte, directly immobilized, or indirectly immobilized via conjugation to a large “carrier” molecule, to the porous support.

If there is no analyte in the sample, the ligand on the tracer can bind to the binder when it reaches to the binder site on the support and the bound complex is detectable at the binding site. If there is analyte in the sample, the ligand can bind to the free analyte in the sample before the analyte and the tracer reach to the binder and the ligand will not bind to the binder since the binding groove on the ligand is occupied by the analyte. Therefore, the tracer will not stop at the binder site (competition test).

Alternatively, the analyte can be a large molecule that contains same or different structures at distanced locations on the large molecules so that the binder, in this case it is antibody or other chemical that can specifically bind to one of the structures on the large molecule, will capture the moving analyte in the capillary flow, whereas the ligand on the tracer, in this case it is antibody (or other chemical) that can specifically bind to another type structure on the large molecule will bind to the large molecule, resulting a “sandwich” complex at the binder site and the bound complex is detectable at the binding site.

In the case that the analyte is non-existent in the sample, or the content of the analyte is too low, the ligand on the tracer will not form the complex with the binder since the bridging analyte is not available, therefore not detectable, or the formed complex is too little to be detectable (Sandwich method).

It has been known that in order to carry out this test to produce adequate results, there must be certain volume of samples so that sufficient amount of analyte will flow by capillary force to finish the test. Consequently, if collected sample was less than the sufficient amount, then it was diluted with a buffer and a big portion of the sample pad on the lateral flow assay was dipped into the diluted sample. Same thing was done with dried chemical samples. This additional process of diluting the sample can potentially lead the result further away from the desired accuracy as the analyte can get lost in the dilution.

This invention follows the same logic and nature of other lateral flow assays, but improves the method of producing accurate results from small volume of a sample, whether it is liquid or dry chemical, without the need of diluting it. The materials that compose the test strip do not have to be different from that of other test strips. A trace amount of sample, as little as 15 microliters, up to 500 microliters, is collected on a sample pad 5 and is put vertically in a container containing the second reagent. The reagent does not necessarily have to be restricted to a particular sort, but it can include water or other solutions with pH ranging from 3.0 to 10.0 or viscosity of ranging values. This reagent is necessary to run the assay test. In order to avoid diluting the sample, the amount of reagent should be enough to only marginally (about 1 mm or less) cover the tip of the sample pad. This would still be enough to allow the capillary force to push the reagent up along with the analyte in the sample to finish the test, allowing the analyte to bind to the ligand or the binder material that has previously been immobilized on the membrane. The minimum or no dilution allows the test to produce maximum detectability and sensitivity as the analyte would have been preserved, generating results that are more accurate and precise.

The removal of dilution process allows the collection of a sample in small amount. Additionally, due to the small amount of sample required, sample collection time is significantly reduced. Other forms of lateral flow assay require that the sample be collected through a secondary device and then transferred to the sample pad. This method has the risk of losing some of the sample, therefore losing the analyte, to the device during the transfer, consequently requiring the collection of more than necessary amount of the sample. With this invention, collecting sample can be done directly through the sample pad, either by swabbing the mouth or quickly sweeping the surface of an object. This assay then can immediately be put into the second reagent to carry out the test. The method of collection does not need to be limited to swabbing or sweeping. The sample can be collected from any source of suspected object or person, as long as the analyte can be obtained for testing.

This is a valuable application in the field of onsite testing of drug abuse. Urine samples are not necessary, allowing the drug test to be done onsite, preventing the attempt to contaminate or dilute the sample, and protecting the privacy of the subject. The short collection time allows the sample to be collected in seconds. Particular reagent is also not necessary since the choice of second reagent is flexible.

What is claimed is:

1. A system of performing lateral capillary flow assay, comprising:
a sample housing unit that exposes a sample collecting portion of the sample testing unit that is utilized for direct sample collection with the sample testing unit;
a sample testing unit comprising:
a sample receiving area to receive the sample analyte,
an indicator holding area to temporarily hold at least one type of indicator material that binds with a corresponding target analyte in the sample liquid to form an analyte-indicator complex that flows across the analyte testing unit under capillary action,
at least one binding area to immobilize at least one type of binder material configured to bind with the at least one type of indicator material, at least one analyte, or both the at least one analyte and the at least one type of indicator material, wherein a presence of the corresponding type of indicator material at the at least one binding area indicates an absence of a corresponding type of target analyte, and
a validation area comprising a ligand or a binder material that selectively binds to the at least one type of indicator material to confirm that the at least one type of indicator material properly flowed across the analyte testing unit under capillary action,
a sample well shaped to hold the sample testing unit in a vertical position, comprising:
an aqueous solution that covers only 1 millimeter of the tip of the sample receiving area that is loaded with the analyte to be tested.

2. The system of claim 1 wherein the analyte can be dried chemicals or aqueous solutions from life body or from surface of objects.

3. The system of claim 1 wherein the analyte can be added or manually swiped to the sample receiving area of the sample testing unit.

4. The system of claim 1 wherein the sample receiving area can be comprised of an absorbent material that is connected to another absorbent material with an overlap.

5. The system of claim 1 wherein the analyte can be protein(s), antibody, enzyme(s), glycoprotein(s), peptide(s), small molecule chemicals, nucleotides, DNA, RNA, lipid(s) and carbohydrates, as well as the metabolites of above mentioned components.

6. The system of claim 1 wherein the detection method can be any of the method of lateral flow immunoassay, lateral flow chemical assay, microfluidic immunoassay, microfluidic chemical assay platforms, and the assay marker can be colorimetric, fluorescent, chemoluminescent.

7. The system of claim 1 wherein after the sample loading to the sample receiving area, at least 1 millimeter of one end of the sample receiving area will start to contact with at least 0.05 milliliter of aqueous solution contained in the sample well.

8. The system of claim 1 wherein the aqueous solution comprising:
water, mixture organic and inorganic solutions at variable ratios, with the water or an inorganic solution content being more than 50%, and
chemical buffers with pH range 3 to 10 and mole concentration between 0 to 2 moles.

9. The system of claim 7 wherein the test strip(s) is (are) tested in vertical position.

10. The system of claim 7 wherein at least 10 (ten) mm of the sample receiving area does not contact the aqueous solution in the sample well.
